(19) United States
(12) Patent Application Publication
(10) Pub. No.: US 2026/0204370 A1
(43) Pub. Date: Jul. 16, 2026

(54) SYSTEMS AND METHODS FOR DETERMINING RESOURCE INDEXES USING RULE-BASED INFERENCE AND LARGE LANGUAGE MODELS

(71) Applicant: Patient Voice AI, Inc., Prosper, TX (US)

(72) Inventors: Jordi Torras, Allen, TX (US); Arnau Torras, Allen, TX (US)

(21) Appl. No.: 19/097,510

(22) Filed: Apr. 1, 2025

Related U.S. Application Data
(60) Provisional application No. 63/744,547, filed on Jan. 13, 2025.

(51) Int. Cl. G16H 10/60; G06F 40/205

(52) U.S. Cl. CPC G16H 10/60; G06F 40/205

(57) ABSTRACT

Provided herein are system and methods for determining resource indexes. In some embodiments, the system and methods include parsing patient medical documents into different sections; determining, using a language model applied to the sections, one or more medical conditions of a patient; placing the one or more medical conditions into a standardized data structure; determining a resource index score for the patient using a rules-based inference engine applied to the standardized data structure; and generating, using the language model, an explanation of the index score based on the rules-based inference engine.

### CROSS-REFERENCE TO RELATED APPLICATION(S)

This application claims priority to U.S. Provisional Application No. 63/744,547 filed on Jan. 13, 2025, the benefit of which is claimed and the disclosure of which is incorporated herein in its entirety.

### FIELD OF THE DISCLOSURE

The present disclosure relates, in general, to resource allocation, and more particularly, to devices, systems, and methods for determining healthcare resource load across a patient population.

### BACKGROUND

The computation of a resource index score such as a Case Mix Index (CMI) is critical for healthcare providers as it serves as an indicator of disease severity and resource requirement, i.e., of the complexity and resource utilization of patient care. Current methods for determining CMI often rely on processes that are prone to inaccuracies, inefficiencies, and lack of transparency.

### BRIEF DESCRIPTION OF THE DRAWINGS

FIG. 1 is an exemplary system of a computing environment where embodiments can be implemented;

FIG. 2 is an exemplary block diagram of an intelligent data storage system, according to one or more embodiments;

FIG. 3 is an exemplary block diagram of a scoring and verification system, according to one or more embodiments;

FIG. 4 is an example flow chart of a method for determining a resource index, according to one or more embodiments;

FIG. 5 is an example flow chart of a method for intelligent storage of a medical record, according to one or more embodiments; and

FIG. 6 is a block diagram of a computer system suitable for implementing one or more components or operations in FIGS. 1-5.

FIG. 7 is a simplified diagram illustrating a neural network structure that may be implemented in resource index engine, according to some embodiments.

FIG. 8 is a diagram illustrating a large language model, according to some embodiments.

### DETAILED DESCRIPTION

The following disclosure provides many different embodiments. Specific examples of components and arrangements are described below to simplify the present disclosure. These are, of course, merely examples and are not intended to be limiting. In addition, the present disclosure may repeat reference terms in the various embodiments. This repetition is for the purpose of simplicity and clarity and does not in itself dictate a relationship between the various embodiments and/or configurations discussed.

In order to provide improved determinations of a resource index, such as a Case Mix Index (CMI), the examples described herein use a combination of rule-based inference engines employing forward or backward chaining logic, large language models for natural language understanding and document content verification. Furthermore, techniques for processing patient documentation in PDF format and utilizing embeddings stored in a vector database for indexing and retrieval are provided. In some embodiments, the system and methods described herein utilize patient documentation in PDF format as input and applies a series of rules to calculate the CMI. Rules may contain arithmetic expressions and logical conditions and can reference other rules, creating a chaining effect. The terminal nodes in the rule chain employ large language models to validate information extracted from the documents. In some embodiments, the system outputs the computed CMI, an explanation of the calculation process, and specific references to the sections and pages of the patient documentation.

With the techniques described herein CMI computation is performed with high precision and provides detailed explanations of its determinations, including specific references to patient documentation. For example, medical records which support the determination of a CMI may be provided to a user. A written explanation may be generated by a large language model that explains the reasons for the determined CMI value. Thus, the techniques described herein significantly reduces manual effort in computing CMI, enhance precision by combining deterministic rules and advanced neural network/AI/LLM techniques, provide detailed explanations and document references, fostering trust and compliance, and efficiently processes large volumes of documents using vectorized embeddings and forward chaining logic.

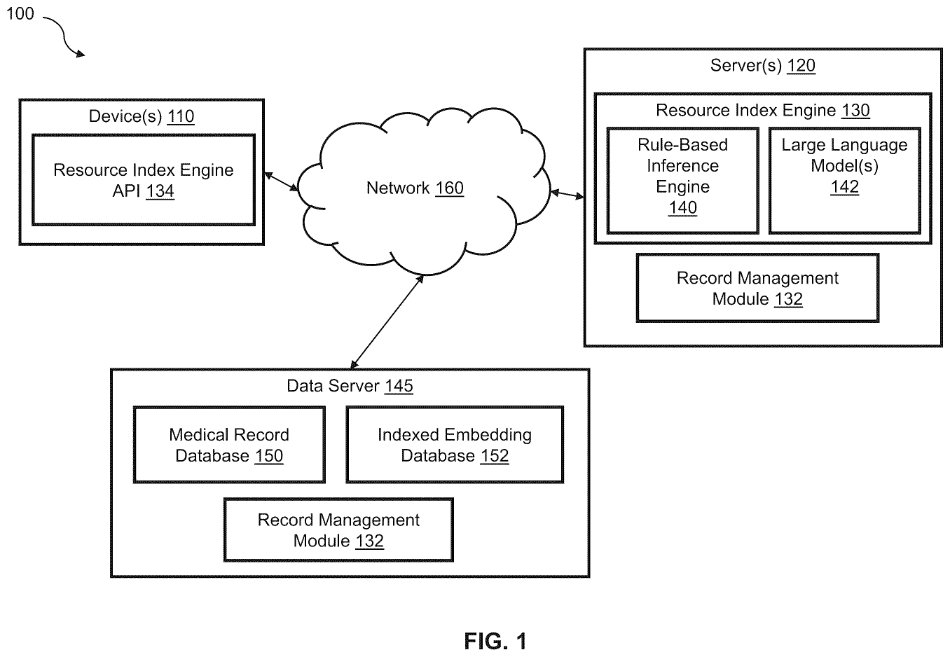

FIG. 1 is an exemplary system 100 where embodiments can be implemented. System 100 may be a computing environment or a computing system. System 100 includes a network 160. Network 160 may be implemented as a single network or a combination of multiple networks. For example, in various embodiments, network 160 may include the Internet or one or more intranets, landline networks, wireless networks, and/or other appropriate types of networks. Network 160 may be a small-scale communication network, such as a private or local area network, or a larger scale network, such as a wide area network. In some embodiments, the network may also include Wi-Fi®, Bluetooth®, and Long-Term Evolution (“LTE”) or other wireless broadband communication technology.

Various components that are communicatively connected to network 160 may be device(s) 110 and server(s) 120. Devices 110 may be portable and non-portable electronic devices under the control of a user and configured to transmit, receive, and manipulate data from server 120 over network 160. Example devices 110 include desktop computers, laptop computers, tablets, smartphones, wearable computing devices, eyeglasses that incorporate computing devices, implantable computing devices, etc.

Server(s) 120 may be electronic devices configured for large scale data processing and service, and may include a physical computer, a data center, a server program that facilitates processing, and the like. Server 120 may include a resource index engine 130 and/or record management module 132. In some embodiments, record management module 132 may be a submodule of resource index engine 130. Resource index engine 130 may be implemented in software, hardware, or a combination of software and hardware. Record management module 132 may be implemented in software, hardware, or a combination of software and hardware. In some embodiments, resource index engine 130 may comprise a rule-based inference engine 140 and/or large language model(s) 142. As used herein, the term “Large Language Model” (LLM) may refer to a neural network based deep learning system designed to understand and generate human languages. An LLM may adopt a Transformer architecture that often entails a significant amount of parameters (neural network weights) and computational complexity. For example, LLM such as Generative Pre-trained Transformer (GPT) 3 has 175 billion parameters, Text-to-Text Transfer Transformers (T5) has around 11 billion parameters. An LLM may comprise an architecture of mixed software and/or hardware, e.g., including an application-specific integrated circuit (ASIC) such as a Tensor Processing Unit (TPU).

In some embodiments, resource index engine 130 applies a series of rules to calculate the CMI for a group of patients. Rules may contain arithmetic expressions and logical conditions and can reference other rules, creating a chaining effect. In some embodiments, rules may include arithmetic expressions which are used to calculate numerical values based on patient attributes or extracted data from medical records. In some embodiments, rules may include logical expressions which define conditions that must be satisfied for a rule to be triggered. In some embodiments, rules may include chained rules, where a rule can reference one or more other rules, enabling complex logical structures. For example, rule 2 may depend on the result of applying rule 1. In some embodiments, resource index engine 130 iteratively evaluates rules until terminal conditions are reached. Terminal nodes may represent conditions requiring processing, e.g., verification or elaboration, by large language model(s) 142.

In some embodiments, large language model(s) 142 may interpret conditions described in natural language, retrieve and/or utilized as input relevant chunks of the patient documentation and structure that information in a standardized way such as a vector database, e.g., indexed embedding database 152 as described below, and validate whether the conditions are satisfied by the content of a document, such as a medical record including patient information. In some embodiments, the output of the resource index engine 130 may produce a CMI value applicable to one or more patients, a detailed explanation of the rule-chaining process, intermediate results, and the logic applied, and specific sections and pages of the patient documentation that contributed to the computation, ensuring transparency and traceability.

Device 110 may include a resource index engine API 134. Resource index engine API 134 may communicate with resource index engine 130 over network 160, provide instructions and data to resource index engine 130 and vice versa. Resource index engine API 134 may display the output of the resource index engine 130, such as the CMI value, explanation for the CMI value generated by the large language model(s) 142, document references that support the determination of the CMI value, and other outputs described herein. Resource index engine API 134 may also receive input such as user feedback that corrects or changes the results of the resource index engine 130, etc.

System 100 may also include a data server 145. Data server 145 may be a database or another large memory storage conducive to storing and retrieving large amounts of data. Data server 145 may store data, including medical records and other documents containing patient information stored in a medical record database 150 and embeddings of the medical records and other documents stored in indexed embedding database 152. The medical record database 150 and indexed embedding database 152 may be accessed from data server 145, downloaded onto device 110 and/or server 120 and used by resource index engine 130. In some embodiments, documents in the medical record database 150 may also be stored in the indexed embedding database 152.

In some embodiments, data sever 145 and/or server(s) 120 may include a record management module 132. In some embodiments, record management module 132 may ingest and process patient documentation in PDF format. For example, PDF documents may be stored in medical record database 150. In some embodiments, record management module 132 may split the documents into manageable chunks suitable for natural language processing by large language model(s) 142. In some embodiments, record management module 132 may generate document embeddings for each chunk using a pre-trained language model (e.g., a pre-trained language model in large language model(s) 142). In some embodiments, document embeddings are stored and indexed in indexed embedding database 152. In some embodiments, metadata may be associated with an embedding stored in indexed embedding database 152. For example, metadata may include the date the record was created, an indication of what large language model(s) 142 was used to create the embedding. Embeddings stored in embedding database 152 are indexed according to one or more schemes to allow for fast and efficient retrieval. For example, embedding database 152 may be a vector database, where individual embeddings are vectors that may be compared to determine a degree of separation.

Although illustrated on data server 145, documents and embeddings in medical record database 150 and indexed embedding database 152 may also be stored locally on the server 120 and be uploaded in whole or in part for display on resource index engine API 134.

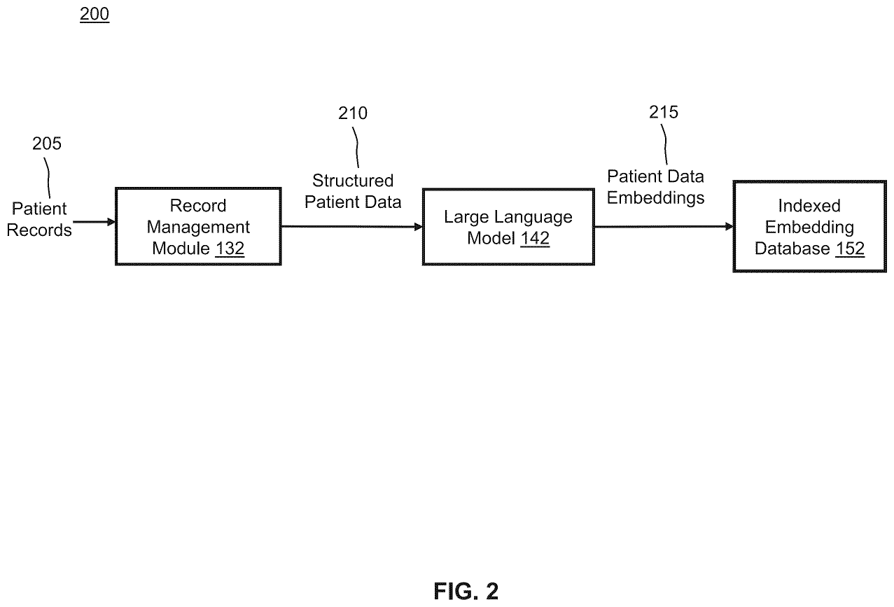

FIG. 2 is an exemplary block diagram of an intelligent data storage system 200, according to one or more embodiments. Intelligent data storage system 200 may ingest and pre-/process patient documentation. For example, patient documentation may be in PDF format and split into manageable chunks suitable for natural language processing by a large language model, generate embeddings for each chunk using a pre-trained language model, store the embeddings and their metadata in a vector database for efficient similarity-based retrieval. In some embodiments, intelligent data storage system 200 may include record management module 132, large language model 142, and indexed embedding database 152.

In some embodiments, record management module 132 may receive patient records 205 as input and provide structured patient data 210 as output. Patient records may include treatments, procedures, diagnoses, conditions, etc., which, for example, may be stored and retrieved from medical record database 150 shown in FIG. 1. Patient records may be in various formations, such as PDF or specialized formats for electronic medical records (EMR). Record management module 132 may parse, segment, or otherwise process the patients records 205 into structured patient data 210 comprising a one or more chunks of the patient records 205.

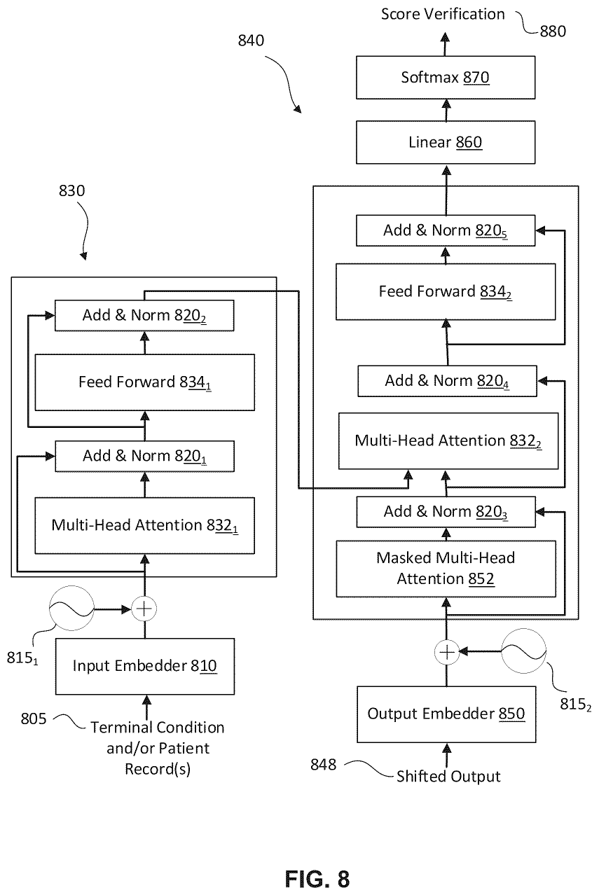

In some embodiment, large language model 142 may receive structured patient data 210 as input and provide patient data embeddings 215 as output. Large language model 142 may include both an embedder, or an encoder, and a decoder, as shown in FIG. 8 and described below. To generate the output patient data embeddings 215, the embedder or encoder of the large language model 142 used. The patient data embeddings 215 may be structured as one or more vector embeddings which are searchable using various metrics such as cosine similarity or the Euclidean distance between a vector and the patient data embeddings 215.

In some embodiments, indexed embedding database 152 may receive patient data embeddings 215 for storage and search. Indexed embedding database 152 allows for the efficient search of patient records for relevant contextual information about a patient. For example, indexed embedding database 152 may be utilized in a retrieval-augmented generation system to evaluate terminal conditions, as described herein. In some embodiments, indexed embedding database 152 may be a vector database or any other database associated with a data structure configured for efficient search.

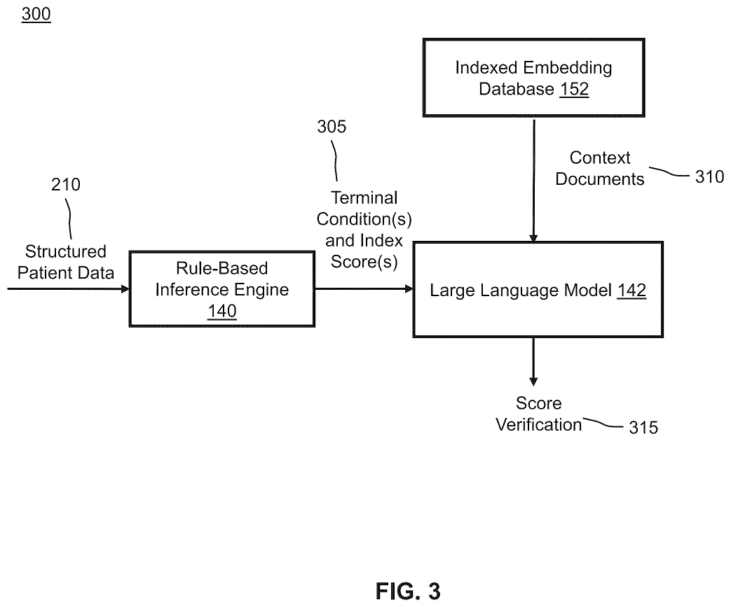

FIG. 3 is an exemplary block diagram of a scoring and verification system 300, according to one or more embodiments. Scoring and verification system 300 may be used to generate index scores and to maximize or minimize the score by verifying if one or more conditions are met. For example, the use of a ventilator in a treatment of a patient may raise a resource index score relative to a similar treatment without a ventilator, so the patient records may be automatically checked to verify whether a ventilator was used in the treatment of the patient. In some embodiments, scoring and verification system 300 may include rule-based inference engine 140, large language model 142, and indexed embedding database 152.

In some embodiments, rule-based inference engine 140 may receive structured patient data 210 as input and output terminal condition(s) and index score(s) 305. Structured patient data may be generated as described above with respect to FIGS. 1-2. As described above, rule-based inference engine 140 generates terminal conditions that represent conditions requiring evaluation. The rule-based inference engine may use the structured patient data for to evaluate a series of rules, which lead to one or more terminal conditions. Then the large language model 142 may verify or elaborate on the terminal condition.

For examples of the impact of terminal conditions on the resource index score, e.g., CMI score, reference is made to the following table:

TABLE 1

| | | | | Patient | Number | |
|---|---|---|---|---|---|---|
| | | | | functional | of | |
| Nursing | | | | ability | restora- | Nurs- |
| case- | | | | and | tive | ing |
| mix | Extensive | Clinical | Depres- | mobility | nursing | CMI |
| group | services | conditions | sion | score | services | score |
| ES3 | Tracheostomy | | | 0-14 | | 4.06 |
| | & Ventilator | | | | | |
| ES2 | Tracheostomy | | | 0-14 | | 3.07 |
| | or Ventilator | | | | | |
| ES1 | Infection | | | 0-14 | | 2.93 |
| HDE2 | | Serious | Yes | 0-5 | | 2.40 |
| | | medical | | | | |
| | | conditions | | | | |
| | | e.g. | | | | |
| | | comatose, | | | | |
| | | septicemia, | | | | |
| | | respiratory | | | | |
| | | therapy | | | | |
| HDE1 | | Serious | No | 0-5 | | 1.99 |
| | | medical | | | | |
| | | conditions | | | | |
| | | e.g. | | | | |
| | | comatose, | | | | |
| | | septicemia, | | | | |
| | | respiratory | | | | |
| | | therapy | | | | |
| HBC2 | | Serious | Yes | 6-14 | | 2.24 |
| | | medical | | | | |
| | | conditions | | | | |
| | | e.g. | | | | |
| | | comatose, | | | | |
| | | septicemia, | | | | |
| | | respiratory | | | | |
| | | therapy | | | | |
| HBC1 | | Serious | No | 6-14 | | 1.86 |
| | | medical | | | | |
| | | conditions | | | | |
| | | e.g. | | | | |
| | | comatose, | | | | |
| | | septicemia, | | | | |
| | | respiratory | | | | |
| | | therapy | | | | |

For a first example, consider the case-mix groups ES1, ES2, and ES3 for extensive services. Rule-based inference engine 140 may determine that a patient received a tracheostomy. However, to determine the final CMI score for the patient depends on if the patient was placed on a ventilator (compare rows for ES3 and ES2). Therefore, the terminal condition generated by the rule-based inference engine 140 is whether or not a ventilator was used. The terminal condition may be embedded, e.g., using the encoder/embedding of LLM 142, and the indexed embedding database 152 searched for records relevant for determining use of a ventilator.

For a second example, consider the case-mix groups HDE1, HDE2, HBC1, and HBC2. Rule-based inference engine 140 may determine that a patient has septicemia and that their functional mobility score is 1. Therefore, the CMI score would be 2.40 or 1.99, depending on the terminal condition, i.e., whether or not the patient has depression. The terminal condition may be embedded, e.g., using the encoder/embedding of LLM 142, and the indexed embedding database 152 searched for records relevant for determining if the patient is currently diagnosed with depression.

For a third example, a patient may have had a tracheostomy placed and septicemia. When only a single index score may be used for a patient in determining a resource allocation, the rule-based inference engine may determine that a score of either 3.07—tracheostomy without ventilator—or 2.40—a patient with depression and septicemia. In this example, the rule-based inference engine 140 may select the higher of the two scores. In some embodiments, when a patient may be assigned multiple scores, other methods of combining the scores may be used, e.g., mean, median, or other averages.

In some embodiments, large language model may receive terminal condition(s) and index score(s) 305 and context documents 310 (e.g., as in the third example above, the patient records indicating a diagnosis of depression and any other patient records used by the rule-based inference engine 140 to arrive at the terminal conditions) as input and output a score verification 315. Context documents 310 may be retrieved from indexed embedding database 152 based on an embedding of the terminal condition. For example, large language model 142 may generate an embedding of the terminal condition and/or portions of the structured patient data, and the generated embedding may be used to search the indexed embedding database 152 for one or more relevant patient records. Using the relevant patient records and/or the terminal condition, large language model 142 may generate a score verification 315. Score verification 315 may include an output that answer a question associated with the terminal condition. For example, returning to the ventilator example, the output may be “a ventilator was used to treat the patient on [DATE]; see [Patient Records] for the indication that a ventilator was used.” In this way, the large language model 142 identifies the source of information that supports the evaluation of the terminal condition.

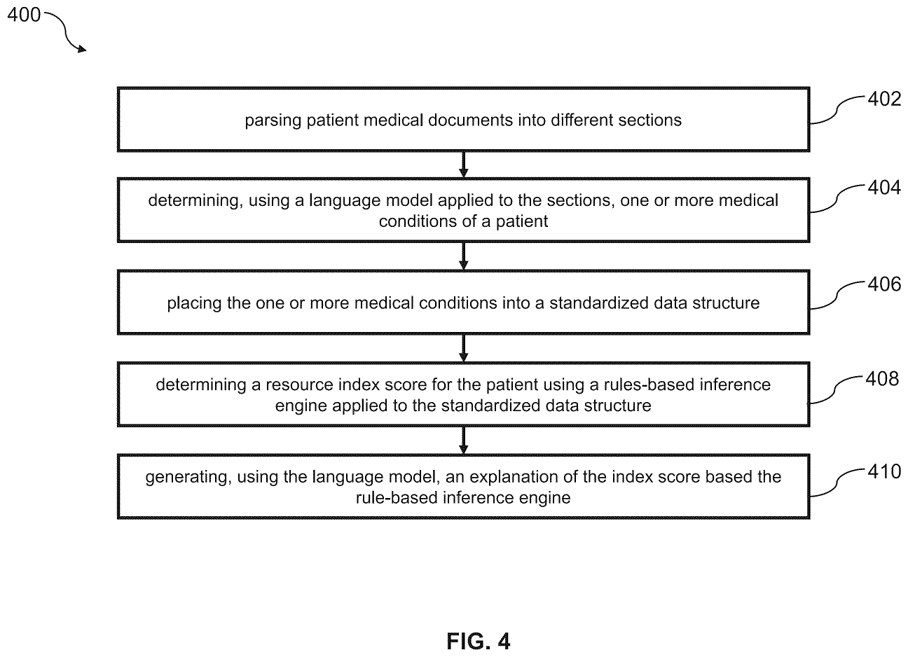

FIG. 4 is an example flow chart of a method for determining a resource index score, according to one or more embodiments. One or more of the processes of method 400 may be implemented, at least in part, in the form of executable code stored on non-transitory, tangible, machine-readable media that when run by one or more processors may cause the one or more processors to perform one or more of the processes. In some embodiments, method 400 corresponds to all or a portion of the operation of the resource index engine 130 as depicted in FIG. 1. Method 400 includes a number of enumerated steps, but aspects of the method 400 may include additional steps before, after, and in between the enumerated steps. In some aspects, one or more of the enumerated steps may be omitted, performed concurrently or performed in a different order.

At operation 402, patient medical records (e.g., patient records 205 in FIG. 2) are parsed into sections or chunks (e.g., structured patient data 210 in FIGS. 2-3). For example, one or more patient records in the form of PDF documents may be divided into separate sections or chunks in a way that makes analysis of those chunks with a large language model more effective. Patient records may be retrieved from a database, e.g., medical record database 150.

At operation 404, determine, using a large language model, a set of medical conditions associated with the user. In some embodiments, record management module 132 may comprise a large language model which is the same as or similar to large language model 142. For example, it may be determined that a user has had a tracheostomy and is on a ventilator by analyzing the various sections or chunks.

At operation 406, place the determined medical conditions into a standardized data structure (e.g., structured patient data 210). For example, the record management module 132 may generate structured patient data from the medical conditions described in patient records 205. For example, the data structure may indicate whether the patient has or does not have any of hundreds of identified medical conditions. The data structure is standardized in that the data for each patient is placed within the same type of data structure. The data structure may be designed for analysis by a rule-based inference engine.

At operation 408, determine a resource index score using a rule-based inference engine (e.g., rule-based inference engine 140). In some embodiments, the rule-based inference engine applies a set of expressions to the standardized data structure for the patient. The set of expressions may be based on a medical insurer's rules for payment. For example, a medical insurer or government medical benefit provider may use a resource index score that relates to the intensiveness of the medical resources required to provide treatment or care. The rules-based inference engine may use a set of expressions that match the medical insurer's or benefit provider's rules for determining resource intensiveness. The rules for calculating the resource index score may be based, for example, on a Patent Driven Payment Model (PDPM) provided by an insurer or government benefit provider. For example, some benefit providers use a Case Mix Index (CMI) to determine the resource requirements for treatment or care and provide compensation accordingly. Thus, the rules-based inference engine can use the patient's medical conditions as derived from the standardized data structure to determine a resource index score (e.g., a CMI).

In some examples, the inference engine may use backward chaining logic. For example, the rules-based inference engine may look at the highest possible index score, and see if it can satisfy all the conditions for that highest possible index score using the standardized data structure for a particular patient. If it cannot meet the conditions for that score, it moves on to the next highest possible index score to see if it can satisfy the conditions for that score. It does this until it finds a match. It may then continue to find certain conditions that will boost that score.

In some examples, the engine may use forward chaining logic by analyzing the medical conditions represented in the data structure for the patient and applying rules to reach a certain resource index score.

The rules used by the rules-based inference engine may be managed or updated by an administrator. The rules may correspond to the criteria set out in a PDMP used by an insurer or government benefit provider. In other words, the rules may be translations of the criteria documented in a PDMP.

In some embodiments, rule-based inference engine generates a terminal condition that is unable to verify based on the structured patient data. In this way, the output of the rule-based inference engine is one or more terminal conditions requiring further evaluation, e.g., using a large language model.

At operation 410, generate, using the language model 142, an explanation of the index score based on a rule applied by the rule-based inference engine. In some embodiments, a language model 142 may use patient information from the standardized data structure and the applied rules used to calculate the CMI value and to summarize how the CMI value was reached. In some embodiments, the chunks of documents used to support the CMI value may be shown or referenced to a user through user device 110. In some embodiments, a user may be asked to review the output of the large language model and verify its accuracy. A user may identify errors and ask the that the score be recalculated with guidance or correction from the user.

In some embodiments, large language model 142 may evaluate the terminal conditions generated by the rule-based inference engine. After the large language model has evaluated the terminal condition, the score may be generated based on the evaluation.

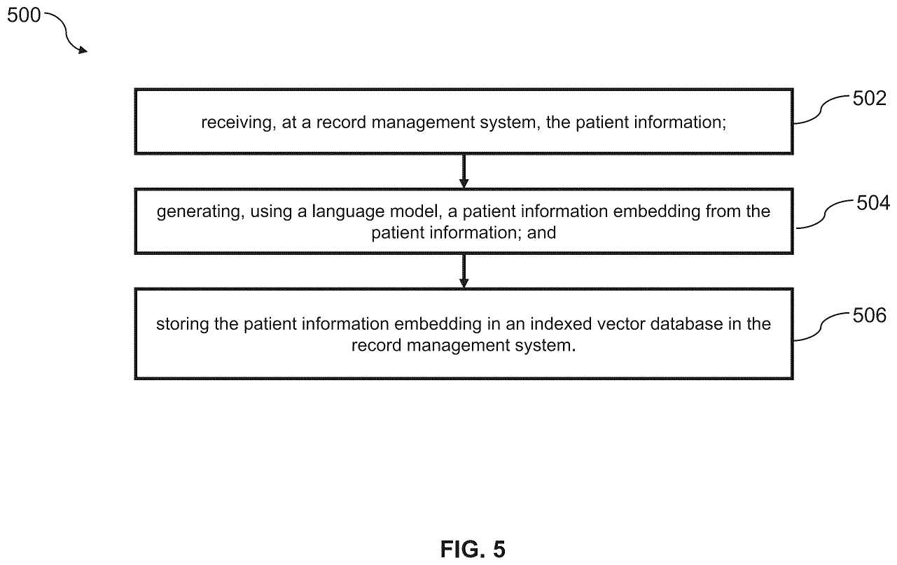

FIG. 5 is an example flow chart of a method for intelligent storage of a medical record, according to one or more embodiments. One or more of the processes of method 500 may be implemented, at least in part, in the form of executable code stored on non-transitory, tangible, machine-readable media that when run by one or more processors may cause the one or more processors to perform one or more of the processes. In some embodiments, method 500 corresponds to all or a portion of the operation of the resource index engine 130 as depicted in FIG. 1. Method 500 includes a number of enumerated steps, but aspects of the method 500 may include additional steps before, after, and in between the enumerated steps. In some aspects, one or more of the enumerated steps may be omitted, performed concurrently or performed in a different order. In some embodiments, methods 400 and 500 may be combined into one method, such that patient records are intelligently stored and a score is calculated based on those patient records.

At operation 502, receive, at a record management system (e.g., server(s) 120 or data server 145 including record management module 132), the patient information. For example, patient information may be contained in medical records. Medical records may indicate various treatments, procedures, diagnoses, conditions, etc. about a patient. In some embodiment, patient information may be stored in medical record database 150.

At operation 504, generate, using a language model 142, a patient information embedding from the patient information. In some embodiments, patient information embedding may be a vector embedding generated by the language model 142.

At operation 506, store the patient information embedding in an indexed vector database 152 in the record management system (e.g., data server 145 including record management module 132). This indexed vector database 152 may store standardized data structures that represent patient medical conditions as described with respect to FIG. 4.

In some examples, the vectors may be stored as dense vectors. In further examples, multiple vectors may be compared using cosine similarity techniques. By determining which portions of the medical records have more variability versus being highly similar can determine which areas of the documents should be focused on by the rules-based inference engine.

In some embodiments, methods 400 and 500 may be combined, such that both document ingestion, chunking, and embedding, are combined with the determination of a CMI value. In some embodiments, a combined methods includes: a document ingestion module (e.g., record management module 132 and/or language model(s) 142) configured to split patient documentation in PDF format into chunks and generate embeddings for indexing in a vector database 152; a rule-based inference engine 140 employing forward chaining to evaluate rules, wherein the rules comprise arithmetic expressions, logical conditions, and references to other rules; a large language model module configured to process terminal nodes in the rule chain by interpreting conditions, retrieving document chunks, and validating information using natural language processing (e.g., using language model(s) 142); and an output generation module (e.g., the output function of resource index engine 130) configured to provide the computed CMI, an explanation of the computation process, and references to specific sections and pages of the patient documentation.

In some embodiments, a vector database 152 stores embeddings and metadata for efficient similarity-based retrieval.

In some embodiment, the rule-based inference engine 140 supports rule chaining to enable complex logical structures.

In some embodiment, the large language model 142 module utilizes a pre-trained language model to validate conditions described in natural language. In some embodiments, a method for computing Case Mix Index (CMI) of patients, comprises: preprocessing (e.g., using record management module 132) patient documentation in PDF format to generate embeddings and store them in a vector database (e.g., indexed embedding database 152); evaluating a plurality of rules using a rule-based inference engine 140 with forward chaining logic; validating terminal conditions using large language models 142 to analyze retrieved document content; and generating an output comprising the computed CMI, a detailed explanation, and document references.

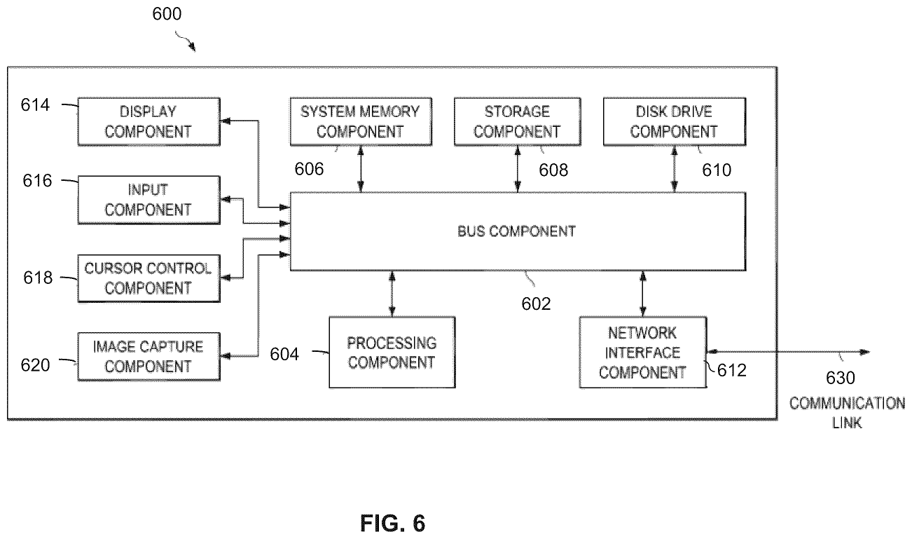

FIG. 6 is a block diagram of a computer system 600 suitable for implementing various methods and devices described in FIGS. 1-5.

In accordance with various aspects of the present disclosure, the computer system 600, such as a network server or a mobile communications device, may include a bus component 602 or other communication mechanisms for communicating information, which interconnects subsystems and components, such as a computer processing component 604 (e.g., processor, micro-controller, digital signal processor (DSP), etc.), system memory component 606 (e.g., RAM), static storage component 608 (e.g., ROM), disk drive component 610 (e.g., magnetic or optical), network interface component 612 (e.g., modem or Ethernet card), display component 614 (e.g., cathode ray tube (CRT) or liquid crystal display (LCD)), input component 616 (e.g., keyboard), cursor control component 618 (e.g., mouse or trackball), and image capture component 620 (e.g., analog or digital camera). In one implementation, disk drive component 610 may comprise a database having one or more disk drive components.

In accordance with aspects of the present disclosure, computer system 600 performs specific operations by the processing component 604 executing one or more sequences of one or more instructions contained in system memory component 606. Such instructions may be read into system memory component 606 from another computer readable medium, such as static storage component 608 or disk drive component 610. In other aspects, hard-wired circuitry may be used in place of (or in combination with) software instructions to implement the present disclosure. In some aspects, the various components of the resource index engine 130 and record management module 132 may be in the form of software instructions that can be executed by the processing component 604 to automatically perform context-appropriate tasks on behalf of a user.

Logic may be encoded in a computer readable medium, which may refer to any medium that participates in providing instructions to the processing component 604 for execution. Such a medium may take many forms, including but not limited to, non-volatile media and volatile media. In one aspect, the computer readable medium is non-transitory. In various implementations, non-volatile media includes optical or magnetic disks, such as disk drive component 610, and volatile media includes dynamic memory, such as system memory component 606. In one aspect, data and information related to execution instructions may be transmitted to computer system 600 via a transmission media, such as in the form of acoustic or light waves, including those generated during radio wave and infrared data communications. In various implementations, transmission media may include coaxial cables, copper wire, and fiber optics, including wires that comprise bus 602.

Some common forms of computer readable media include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, carrier wave, or any other medium from which a computer is adapted to read. These computer readable media may also be used to store the programming code for the resource index engine 130 and record management module 132 discussed above.

In various aspects of the present disclosure, execution of instruction sequences to practice the present disclosure may be performed by computer system 600. In various other aspects of the present disclosure, a plurality of computer systems 600 coupled by communication link 630 (e.g., a communications network, such as a LAN, WLAN, PTSN, and/or various other wired or wireless networks, including telecommunications, mobile, and cellular phone networks) may perform instruction sequences to practice the present disclosure in coordination with one another.

Computer system 600 may transmit and receive messages, data, information and instructions, including one or more programs (i.e., application code) through communication link 630 and network interface component 612. Received program code may be executed by processing component 604 as received and/or stored in disk drive component 610 or some other non-volatile storage component for execution. The communication link 630 and/or the network interface component 612 may be used to conduct electronic communications between the resource index engine 130 and record management module 132, as described in FIG. 1, and external devices, for example with the device 110 depending on where the resource index engine 130 and record management module 132 are implemented.

In some embodiments, display component 614 may display the output from the resource index engine 130 to a user operating user device 110, similar to computer system 600. For example, a user may view a CMI value, and explanation of the reasoning used to generate the CMI values, and references to document sections supporting the reasoning and final CMI value.

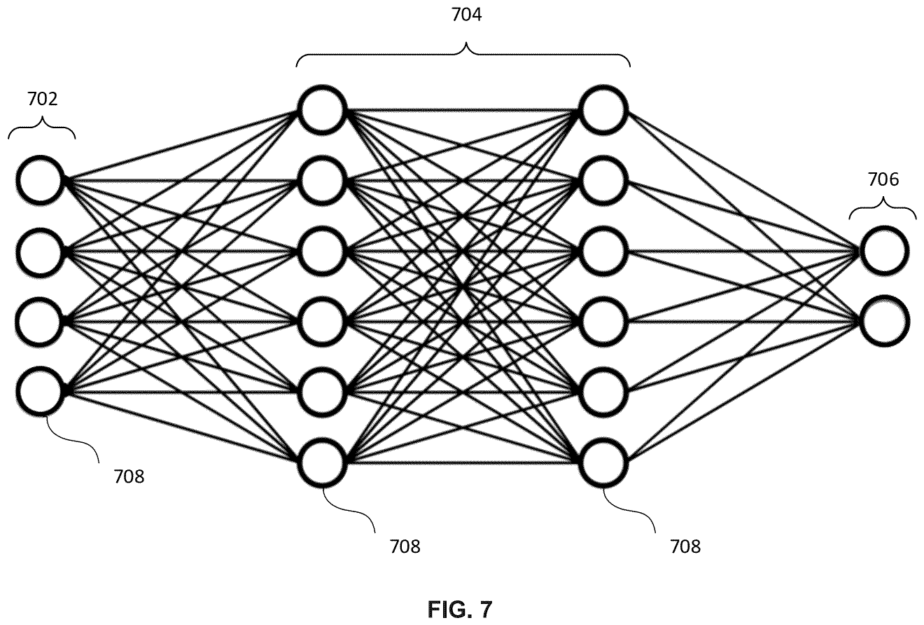

FIG. 7 is a simplified diagram illustrating a neural network structure that may be implemented in resource index engine 130, according to some embodiments. Resource index engine 130 may include a perception neural network, a feed forward neural network, a multilayer perceptron network, a convolutional neural network, a radial basis functional neural network, a recurrent neural network, an LSTM (Long Short-Term Memory) network and the like. In some instances, resource index engine 130 may be implemented as one or more generative pre-trained transformer (GPT) models, large language models (LLMs), or a bi-directional encoder representations from transformers (BERT) models.

Resource index engine 130 may comprise a neural network architecture. The example neural network architecture may comprise an input layer 702, one or more hidden layers 704 and an output layer 706. The resource index engine 130 may be built as a collection of connected units or nodes, referred to as neurons 708. Each layer 702, 704, or 706 may comprise the same or different number of neurons or nodes 708, with neurons between layers being interconnected according to a specific topology. Each neuron 708 may be associated with an adjustable weight. The neurons 708 may be aggregated into layers 702, 704, 706 such that different layers may perform different transformations on the respective input to generate a transformed output, which is an input for the subsequent layer. Further, different layers in resource index engine 130 may be combined into their own neural network models, such that an output layer of one neural network model is an input into the next neural network model until a final output layer 706 is reached.

Input layer 702 receives input data, such as terminal conditions and/or medical records from rule-based inference engine 140 and/or indexed embedding database 152. The number of nodes (neurons) in the input layer 702 may be determined by the dimensionality of the input data (e.g., the length of a vector of a given example of the input). Each node 708 in the input layer 702 may represent a feature or attribute of the input. In some embodiments, input layer 702 may be an embedding layer that may generate embeddings from input data. For example, words or tokens input data may be converted into vectors of fixed size called embedding vectors. The embedding vectors are mapped into a high-dimensional space. Additionally, positional encodings are added to the embedding vectors that may preserve the order of words in the input. Thus, each word and/or number in the input data may be transformed into embedding vectors, with the position each word and/or number maintained using the positional embeddings.

The hidden layers 704 are intermediate layers located between the input and output layers 702, 706 of the resource index engine 130. Although three hidden layers 704 are shown, there may be any number of hidden layers in the resource index engine 130. Hidden layers 704 may extract and transform the input data through a series of weighted computations and activation functions associated with individual neurons.

For example, the resource index engine 130 may receive prompts and data (e.g., terminals conditions and/or patient records) at input layer 702 and generate prompt-responsive text or classifications (e.g., an evaluation of the terminal condition to device a final score for a patient) in an output of output layer 706. To perform the transformation, each neuron 708 receives input signals (which may be input, such as terminal conditions and/or patient records, to resource index engine 130 or output of the preceding layer), performs a weighted sum of the inputs according to weights assigned to each connection and then applies an activation function associated with the respective neuron 708 to the result. The output of the neuron is passed to the next layer of neurons or serves as the final output of the network. The activation function may be the same or different across different layers 702, 704, 706, and may be different at neurons 708 within each layer. Example activation functions include but are not limited to Sigmoid, hyperbolic tangent, Rectified Linear Unit (ReLU), Leaky ReLU, softmax, and/or the like. In this way, input data received at the input layer 702 is transformed by hidden layers 704 into different values indicative of data characteristics corresponding to a task that the resource index engine 130 has been trained to perform. Furthermore, the hidden layers may be organized and connected into larger network structures as described below.

The output layer 706 is the final layer of the neural network structure. It produces the network's output or prediction based on the computations performed in the preceding layers (e.g., 702, 704). The number of nodes in the output layer depends on the nature of the task being addressed. For example, in a binary classification problem, the output layer may consist of a single node representing the probability of belonging to one class. In a multi-class classification problem, the output layer may have multiple nodes, each representing the probability of belonging to a specific class. In the embodiments discussed herein, an output of output layer 706 may be an evaluation of one or more terminal conditions determined by rule-based inference engine 140.

FIG. 8 is a diagram of a large language model 800, according to some embodiments. In some embodiment, large language model (LLM) may be used for terminal condition evaluation, score verification and explanation, and patient record embedding. The LLM may receive a terminal condition(s) from rule-based inference engine 140, output from other models in resource index engine 130, and/or patient information as described herein.

The LLM model may include an input embedder 810. Input embedder 810 may be part of input layer 702. Input embedder 810 may receive input text, such as a patient records and/or terminal condition(s) 805, tokenize the input text into tokens, and generate embedding vectors for the tokens that capture semantic and syntactic information from the input text. There may be one embedding vector for one token, where a token may represent a word in the input text. In some instances, positional encodings 815_1 may be added to the embedding vectors to provide information of the positions of the tokens in the input text with respect to other tokens.

In some embodiments, one or more hidden layers 704 may further be combined into layers and/or blocks. Example layers may be an encoder 830, a decoder 840, a linear layer 860, and a softmax layer 870. In a non-limiting embodiment, encoder 830 may include a multi-head attention layer 832_1, one or more normalization layers 820, such as normalization layers 820_1 and 820_2, and a feed forward network 834_1. Decoder 840 may include a masked multi-head attention layer 852, one or more normalization layers 820, such as normalization layers 820_3, 820_4, and 820_5, multi-head attention layer 832_2, and feed-forward layer 834_2. The encoder 830 and decoder 840 may comprise transformer blocks. Further, the outputs of one layer may be inputs into the subsequent layer as shown in FIG. 8.

Encoder 830 may receive the embedding vectors from input embedder 810 and pass the embedding vectors through multi-head attention layer 832_1, normalization layers 820_1 and 820_2 and feed forward layer 834_1 to generate hidden states that include the context and meaning of the input text. In some embodiments, these hidden states are stored in the indexed embedding database 152 as described herein. The multi-head self-attention layer 832_1 may focus on different embedding vectors and identify the importance of different tokens in the input text. The feed forward layer 834_1 may include two linear layers, with each layer including activation functions at its neurons. The neurons of each linear layer of feed forward layer 834_1 may receive input from all neurons of the previous linear layer. The feed forward layer 834_1 may capture interactions between tokens in input text. Normalization layers 820_1 and 820_2 may receive the output of the previous layer as input, e.g., the output multi-head self-attention layer 832_1 and feed forward layer 834_1 respectively and normalize the input. Normalizing the input may ensure that the output of a preceding layer has a consistent distribution. The output of encoder 830 may be the output of normalization layer 820_1. As illustrated in FIG. 8, multiple layers in encoder 830 may receive the embedding vectors and the outputs of the preceding layer.

Decoder 840 may receive an output of encoder 830 and embedding vectors of output embedder 850. Output embedder 850 may receive input text, which may be a shifted output 848 of the LLM. Shifted output 848 may be output 880 which includes score verification, score explanation, and terminal condition evaluation. Output embedder 850 may convert the shifted output 848 into tokens, and generate the embedding vectors from the tokens. In some instances, positional encodings 815_2 may be added to the embedding vectors to provide information on the positions of the tokens in the shifted output 848 with respect to other tokens.

Masked multi-head attention layer 852 and normalization layer 820_3 of decoder 840 may receive the embedding vectors of the shifted output 848. Masked multi-head attention layer 852 may be a variant of multi-head attention layers 832 where the prediction of output tokens depends on previous tokens because the embedding vectors that correspond to future tokens are masked. The output of the masked multi-head attention layer 852 may also be fed into normalization layer 820_3. Normalization layer 820_3 may normalize its input and ensure that the embedding vectors of the shifted output 848 and output of the masked multi-head attention layer 852 have a consistent distribution.

The multi-head attention layer 832_2 may receive the output of encoder 830 and output of normalization layer 820_3 and generate an output that focuses on an importance of different tokens in the input text (e.g., terminal conditions and/or patient records 805 and shifted output 848). The output of the multi-head attention layer 832_2 and normalization layer 820_3 may be fed into the normalization layer 820_4. Normalization layer 820_4 may normalize its input, e.g., the output of the multi-head attention layer 832_2 and the output of normalization layer 820_3 to make sure the input has a consistent distribution.

Feed forward layer 834_2 may capture interactions between tokens in input text and shifted output 848 by processing the output of the normalization layer 820_4 as input. Like feed forward layer 834_1, feed forward layer 834_2 may include two linear layers, with each layer including activation functions at their neurons. The neurons of each linear layer of feed forward layer 834_2 may receive input from all neurons of the previous linear layer.

Normalization layer 820_5 may receive the output of feed forward layer 834_2 and output of normalization layer 820_4 as input and normalize the output, which may be the output of decoder 840.

Linear layer 860 may receive the output of decoder 840. Linear layer 860 and softmax layer 870 may be used to generate a probability distribution of a next token in the large language model output 880, which may be a score verification, score explanation, terminal condition evaluation. Linear layer 860 may be a fully connected layer where all neurons of the linear layer receive inputs from a preceding layer, e.g., normalization layer 820_5, and apply linear transformation to the inputs by applying corresponding weights of the neurons and adding bias. The output of linear layer 860 may be an input to softmax layer 870 that generates large language model output 880. Softmax layer may be an output layer 706 in FIG. 7 which generates probability distributions for next tokens, e.g., a word, to be included in large language model output 880. As discussed above, large language model output 880 may be shifted to be fed as input into decoder 840.

Going back to FIG. 7, resource index engine 130 may also be implemented by hardware, software, and/or a combination thereof. For example, resource index engine 130 may comprise a specific neural network structure implemented and run on various hardware platforms, such as but not limited to CPUs (central processing units), GPUs (graphics processing units), FPGAS (field-programmable gate arrays), Application-Specific Integrated Circuits (ASICs), dedicated AI accelerators like TPUs (tensor processing units), and specialized hardware accelerators designed specifically for the neural network computations described herein, and/or the like. Example specific hardware for neural network structures may include, but not limited to Google Edge TPU, Deep Learning Accelerator (DLA), NVIDIA AI-focused GPUs, and/or the like. The hardware may be used to implement the neural network structure is specifically configured based on factors such as the complexity of the neural network, the scale of the tasks (e.g., training time, input data scale, size of training dataset, etc.), and the desired performance.

Resource index engine 130 may be trained by iteratively updating the underlying weights of the neurons 708, etc., bias parameters and/or coefficients in the activation functions associated with neurons 708. The weights may be updated based on a loss function, such as a mean squared estimation error (MSEE), cross-entropy loss, log-loss, and the like. For example, during training, the training data are fed into resource index engine 130 over thousands of iterations. The training data flows through the network's layers 702, 704, 706, with each layer performing computations based on its weights, biases, and activation functions until the output layer 706 produces the output.

The training data may be labeled with an expected output (e.g., a “ground-truth” such as a corresponding ground truth score verification, score evaluation, or terminal condition evaluation). The output generated by the output layer 706 is compared to the expected output from the training data to compute a loss function that measures the discrepancy between the predicted output and the expected output. In some embodiments, the negative gradient of the loss function may be computed with respect to the weights of each layer individually. This negative gradient is computed one layer at a time, iteratively backward from the last layer 706 to the input layer 702 of the resource index engine 130. These gradients quantify the sensitivity of the network's output to changes in the parameters. The chain rule may be applied to efficiently calculate these gradients by propagating the gradients backward (in a back propagation network) from the output layer 706 to the input layer 702.

Parameters of the neural network are updated backwardly from the last layer to the input layer (backpropagating) based on the computed negative gradient using an optimization algorithm to minimize the loss. The backpropagation from the last layer 706 to the input layer 702 may be conducted for a number of training samples in a number of iterative training epochs. In this way, parameters of the resource index engine 130 may be gradually updated in a direction to result in a lesser or minimized loss, indicating the neural network has been trained to generate a predicted output value closer to the target output value with improved prediction accuracy. Training may continue until a stopping criterion is met, such as reaching a maximum number of epochs or achieving satisfactory performance on the validation data. In a multiple neural network embodiment, the neural network models may be trained separately and then combined together and trained as a single resource index engine 130.

Neural network parameters may be trained over multiple stages. For example, initial training (e.g., pre-training) may be performed on one set of training data, and then an additional training stage (e.g., fine-tuning) may be performed using a different set of training data, such as machine-readable code in one or more programming languages. In some embodiments, all, or a portion of parameters of one or more neural-network models being used together may be frozen, such that the “frozen” parameters are not updated during that training phase. This may allow, for example, a smaller subset of the parameters to be trained without the computing cost of updating all the parameters.

Therefore, the training process transforms the neural network into an “updated” trained neural network with updated parameters such as weights, activation functions, and biases. The trained neural network thus improves neural network technology in score evaluation, score verification, and terminal condition evaluation. Other AI generated content may serve as input as described herein, e.g., patient records, etc. In some embodiments, individual modules, e.g., the LLM 142, may implement the neural network structure as described herein.

Once training is complete, the trained resource index engine 130 may enter an inference stage where resource index engine 130 may be used to make predictions on new, unseen data, such as evaluation terminal conditions and verifying scores based on prompts that include various patient medical records.

Where applicable, various embodiments provided by the disclosure may be implemented using hardware, software, or combinations of hardware and software. Also, where applicable, the various hardware components and/or software components set forth herein may be combined into composite components comprising software, hardware, and/or both without departing from the scope of the disclosure. Where applicable, the various hardware components and/or software components set forth herein may be separated into sub-components comprising software, hardware, or both without departing from the scope of the disclosure. In addition, where applicable, it is contemplated that software components may be implemented as hardware components and vice-versa.

Software, in accordance with the disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The present disclosure also introduces an apparatus, which apparatus has been described according to one or more aspects of the present disclosure.

The present disclosure also introduces a system, which system has been described according to one or more aspects of the present disclosure.

The present disclosure also introduces a method, which method has been described according to one or more aspects of the present disclosure.

The present disclosure also introduces an assembly, which assembly has been described according to one or more aspects of the present disclosure.

It is further understood that variations may be made in the foregoing without departing from the scope of the disclosure.

In one or more embodiments, the elements and teachings of the various embodiments disclosed herein may be combined in whole or in part in some or all of said embodiment(s). In addition, one or more of the elements and teachings of the various embodiments disclosed herein may be omitted, at least in part, or combined, at least in part, with one or more of the other elements and teachings of said embodiment(s).

Any spatial references such as, for example, “upper,” “lower,” “above,” “below,” “between,” “bottom,” “vertical,” “horizontal,” “angular,” “upwards,” “downwards,” “side-to-side,”“left-to-right,” “left,” “right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In one or more embodiments, while different steps, processes, and procedures are described as appearing as distinct acts, one or more of the steps, one or more of the processes, or one or more of the procedures may also be performed in different orders, simultaneously or sequentially. In one or more embodiments, the steps, processes, or procedures may be merged into one or more steps, processes, or procedures. In one or more embodiments, one or more of the operational steps in each embodiment may be omitted. Moreover, in some instances, some features of the present disclosure may be employed without a corresponding use of the other features. Moreover, one or more of the embodiments disclosed above, or variations thereof, may be combined in whole or in part with any one or more of the other embodiments described above, or variations thereof.

Although various embodiments have been disclosed in detail above, the embodiments disclosed are exemplary only and are not limiting, and those skilled in the art will readily appreciate that many other modifications, changes, and substitutions are possible in the embodiments without materially departing from the novel teachings and advantages of the present disclosure. Accordingly, all such modifications, changes, and substitutions are intended to be included within the scope of this disclosure as defined in the following claims. In the claims, means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A method for determining a resource index comprising:
parsing patient medical documents into different sections;
determining, using a language model applied to the sections, one or more medical conditions of a patient;
placing the one or more medical conditions into a standardized data structure;
determining a resource index score for the patient using a rule-based inference engine applied to the standardized data structure; and
generating, using the language model, an explanation of the index score based on the rule-based inference engine.

2. The method of claim 1, wherein determining a resource index score further comprises:
determining a terminal condition using the rule-based inference engine applied to the standardized data structure;
evaluating the terminal condition using the language model; and
generating the resource index score based on the evaluation of the terminal condition.

3. The method of claim 1, wherein the explanation of the index score comprises one or more citations to patient records stored in a database.

4. The method of claim 3, wherein the explanation of the index score comprises a verification request for a user.

5. The method of claim 4, further comprising:
receiving a user verification in response to the verification request; and
determining a final resource index score based on the user verification.

6. The method of claim 3, further comprising:
retrieving, from a database, the patient records.

7. The method of claim 1, further comprising:
generating, using the language model, a one or more medical record
embedding from the patient medical documents; and
storing the one or more medical record embedding in an indexed vector database in a record management system.

8. A system configured to determine a resource index comprising:
a memory configured to store instructions for a language model;
a processor coupled to the memory and configured to read the instructions from the memory to cause the system to perform operations, the operations comprising:
parse patient medical documents into different sections;
determine, using the language model applied to the sections, one or more medical conditions of a patient;
place the one or more medical conditions into a standardized data structure;
determine a resource index score for the patient using a rule-based inference engine applied to the standardized data structure; and
generate, using the language model, an explanation of the index score based on the rules-based inference engine.

9. The system of claim 8, wherein the operations further comprising determine a resource index score by:
determining a terminal condition using the rule-based inference engine applied to the standardized data structure;
evaluating the terminal condition using the language model; and
generating the resource index score based on the evaluation of the terminal condition.

10. The system of claim 8, wherein the explanation of the index score comprises one or more citations to patient records stored in a database.

11. The system of claim 10, wherein the explanation of the index score comprises a verification request for a user.

12. The system of claim 11, wherein the operations further comprising:
receive a user verification in response to the verification request; and
determine a final resource index score based on the user verification.

13. The system of claim 10, wherein the operations further comprising:
retrieve, from a database, the patient records.

14. The system of claim 8, wherein the operations further comprising:
generate, using the language model, a one or more medical record
embed from the patient medical documents; and
store the one or more medical record embedding in an indexed vector database in a record management system.

15. A non-transitory machine-readable medium comprising a plurality of instructions, executable by one or more processors, wherein the plurality of instructions are configurable to cause the one or more processors to perform operations comprising:
parse patient medical documents into different sections;
determine, using a language model applied to the sections, one or more medical conditions of a patient;
place the one or more medical conditions into a standardized data structure;
determine a resource index score for the patient using a rule-based inference engine applied to the standardized data structure; and
generate, using the language model, an explanation of the index score based on the rule-based inference engine.

16. The non-transitory machine-readable medium of claim 15, wherein the operations further comprising determine a resource index score by:
determining a terminal condition using the rule-based inference engine applied to the standardized data structure;
evaluating the terminal condition using the language model; and
generating the resource index score based on the evaluation of the terminal condition.

17. The non-transitory machine-readable medium of claim 15, wherein the explanation of the index score comprises one or more citations to patient records stored in a database.

18. The non-transitory machine-readable medium of claim 17, wherein the explanation of the index score comprises a verification request for a user.

19. The non-transitory machine-readable medium of claim 18, wherein the operations further comprising:
receive a user verification in response to the verification request; and
determine a final resource index score based on the user verification.

20. The non-transitory machine-readable medium of claim 17, wherein the operation further comprising:
retrieve, from a database, the patient records.
